(19) United States
(12) Patent Application Publication
(10) Pub. No.: US 2007/0249884 A1
(43) Pub. Date: Oct. 25, 2007

(54) Polyolefin processes with constituent high conversion alkane dehydrogenation in membrane reactors

(75) Inventors: Martin E. Carrera, Naperville, IL (US); Craig W. Colling, Warrenville, IL (US); Michael J. Foral, Aurora, IL (US)

Correspondence Address: INEOS USA LLC, 3030 WARRENVILLE RD, S/650, LISLE, IL, 60532, US

(73) Assignee: Innovenne USA, Lisle, IL (US)

(21) Appl. No.: 11/407,511

(22) Filed: Apr. 20, 2006

(51) Int. Cl. C07C 2/02

(52) U.S. Cl. 585/530

(57) ABSTRACT

Polymerization processes having as constituent parts high conversion membrane reactors, that provide a source of monomer, and subsequent polymerization of the monomer, without passing products of the conversion through an alkane/alkene splitter, are disclosed. Polymers of light alkene hydrocarbons, such as ethylene, propylene and alkenes consisting of up to 6 carbon atoms, are prepared from gaseous feedstreams consisting predominantly of volatile alkane compounds substantially free of dihydrogen and/or dioxygen. Equipment required for separation of alkene products from unreacted alkanes in conventional plants is eliminated because of the high alkane conversions provided in the membrane reactors. Particularly useful are flow reactors comprising dense membranes of multiphasic materials that provide independent, controllable, counter-current transport of hydrogen, electrons and oxygen.

### TECHNICAL FIELD

The present invention relates to olefin oligomerization and/or polymerization processes having as constituent parts high conversion membrane reactors which provide a source of monomer, and subsequent oligomerization and/or polymerization of the monomer, without passing products of the conversion through an alkane/alkene splitter. More particularly the present invention relates to processes preparing polymers of light alkene hydrocarbons, such as ethylene, propylene and alkenes consisting of up to 6 carbon atoms, from gaseous feedstreams consisting predominantly of volatile alkane compounds substantially free of dihydrogen and/or dioxygen. Equipment required for separation of alkene products from unreacted alkanes in conventional plants is eliminated because of the high alkane conversions provided in the membrane reactors. Particularly useful are flow reactors comprising dense membranes of multiphasic materials that provide independent, controllable, counter-current transport of hydrogen, electrons and oxygen.

### BACKGROUND OF THE INVENTION

Alkenes, commonly known as olefins, are used to produce many useful polymers and as components of numerous synthetic chemicals. Ethylene is used in one of several forms of polyethylene, as ethylene glycol to make polyester, in the manufacture of vinyl acetate and vinyl chloride, as a building block for linear alpha olefins, and in the production of styrene. Propylene is used in the synthesis of polypropylene, propylene oxide, acrylonitrile, and cumene. Butadiene is used primarily to make elastomers including styrene-butadiene rubber, neoprene, and nitrile rubber. The olefins/polymers value chain is typically composed of several distinct steps: (1) conversion of hydrocarbons including alkanes into alkenes, (2) in some cases transformation of the alkenes into intermediate products via oxidation, ammoxidation, or alkylation (e.g. acrylonitrile, styrene, and cumene), (3) polymerization or oligomerization into macromolecules, and (4) final device fabrication into end products.

Several commercialized methods are practiced to synthesize olefins. The most industrially significant method is steam cracking. Steam crackers can produce olefins from numerous hydrocarbon feeds including natural gas liquids, light petroleum gases, light paraffinic naphthas, and mixtures thereof. Commercialized steam cracking processes utilize high temperature pyrolysis where these feeds are mixed with steam and heated to temperatures in a range from about 700 to 900° C. Thermodynamic equilibrium limits olefin yield to relatively low amounts. The olefins industry has gotten above this constraint by pushing temperatures up to where free radical mechanisms start to occur. The industry relies on high temperatures and quick contact time so that free radical reactions start and then quickly quenching the reactants to focus the yield pattern on olefins and limit the formation of byproducts. Reactor development in conventional olefins crackers has been oriented toward shorter and shorter contact times with large quench heat exchangers to quickly stop the reactions. More detail regarding the operation, engineering, and optimization of steam cracking may be found in Ullmann's Encyclopedia of Industrial Chemistry.

When an olefin is made from an alkane, commonly known as paraffin, hydrogen is also produced. Thermodynamics dictates the maximum yield of olefins and hydrogen possible at a specific reactor temperature. Chemical conversions approach but do not exceed the thermodynamic equilibrium limit. See for example, U.S. Pat. No. 6,271,431, in the name of Christian Busson, Jean-Pierre Burzynski, Henri Delhomme, and Luc Nougier, describes a reactor that produces ethylene yields higher than those normally obtained in commercial cracking reactors by lowering the temperature and increasing the contact time of the process. Their process approaches but cannot exceed the thermodynamic equilibrium limit.

U.S. Pat. No. 6,111,156, in the name of Michael C. Oballa, David Purvis, Andrzej Z. Krzywicki, and Leslie W. Benum, describes a high temperature, high conversion olefin process that approaches the maximum thermodynamic yield of olefins. The patent describes furnace tubes or coils that have been adapted to operate at temperatures higher than those typically employed in conventional steam crackers (above 1050° C.), thereby increasing conversion. Examples of these adaptations include coatings available from Surface Engineered Products and ceramic tubes including silicon carbide.

There are several problems with this approach to increasing olefin yield. Joining silicon carbide to metals is very difficult and the technology for doing so, and keeping the joint in tact at these temperatures (above 1050° C.), is not well developed. Therefore, this leads to frequent ceramic tube failures and generally unreliable operations. Furthermore, vibrations typically encountered during steam cracking operations can easily damage and destroy silicon carbide tubes at the elevated temperatures described in U.S. Pat. No. 6,111,156. Olefin selectivity is believed to be poor at these elevated temperatures. If the operation of the steam-cracking reactor is too severe, numerous researchers have pointed out that the amount of olefin produced per pound of feed converted can actually level out and even decrease. For example, a kinetic severity factor (KSF) is defined in “Pyrolysis: Theory and Industrial Practice” edited by L. F. Albright and coworkers and published by Academic Press in 1983 that relates reactor residence time, reactor temperature, reactor pressure, quenching, and feedstock type. They show that the concentration of olefins passes through a maximum as KSF is increased. This occurs because secondary reactions that begin to consume olefins play a larger role at high severity. The amount of undesirable byproducts is also understood to be high when steam cracking at the elevated temperatures described in U.S. Pat. No. 6,111,156.

Alternative routes for the production of ethylene, propylene and butylenes have been of interest for many years as an alternative to steam cracking. Note that all of these can approach but not exceed the thermodynamic conversion limit. The most feasible route to the commercial scale on-purpose production of these alkenes has generally been through the catalytic dehydrogenation of the relevant alkane according to the formula
CₙH₍₂ₙ₊₂₎---------->CₙH₍₂ₙ₎+H₂
where n is an integer greater than or equal to 2. Catalytic dehydrogenation reactions are limited by thermodynamic constraints resulting from the highly endothermic nature of the reaction. As reactor temperatures increase above 600° C., side-cracking reactions based on free radical mechanisms can occur, leading to the formation of lighter hydrocarbons and coke. Employing a catalyst reduces the required reaction temperature and thereby largely avoids the formation of free radical species in the reactor. The high costs of the alkane feedstocks (e.g., ethane, propane, etc.) and the capital required for the dehydrogenation processes make it economically desirable to achieve the highest possible selectivity to alkanes and to limit the formation of coke and coke precursors within the dehydrogenation reactor.

As in catalytic reforming, coke formation on and the resulting deactivation of the dehydrogenation catalyst is reduced by the addition of small amounts of dihydrogen to the dehydrogenation reactor feed. Significant research has been devoted to minimizing the coke formation reaction and in studying the kinetics of coke formation. For instance, R. L. Mieville (Studies in Surface Science and Catalysis, vol. 68, Catalyst Deactivation 1991, pp. 151-159) has showed that the rate of coke formation for a Pt/Al₂O₃ catalyst used in reforming obeys the following equation
rcoke=(A)*(1/pH2)*(pfeed^{0.75})*(1/coke)*(exp(−37000/RT))
Where pH₂ is the partial pressure of hydrogen, pfeed is the partial pressure of the hydrocarbon feed, and “coke” relates to the amount of coke already present on the catalyst. This equation shows that the rate of coke formation is inversely proportional to the hydrogen partial pressure. Without the addition of hydrogen, most dehydrogenation catalysts deactivate in a time frame that is not commercially viable. Typically, in catalytic dehydrogenation processes, the amount of hydrogen added with the reactant alkane for coke suppression is balanced against the reduction in equilibrium conversion brought about by the resulting higher hydrogen concentration. Even with hydrogen addition to the reactor feed, some coke is formed on the catalyst and all commercial catalytic dehydrogenation technologies employ a reactor configuration which is designed to include periodic regeneration of the catalyst.

The extent of the conversion of hydrocarbons to olefins in conventional dehydrogenation systems is typically limited by thermodynamic equilibrium. There is a need for processes that overcome this thermodynamic limit. Removal of this thermodynamic limitation would allow higher per-pass conversion of the hydrocarbon to take place, resulting in a more efficient overall process.

One method that can be employed to remove the thermodynamic limitation is to employ oxidative dehydrogenation of the alkane. Oxidative dehydrogenation of ethane to ethylene has been reviewed recently by Dai et al. (Current Topics in Catalysis, 3, 33-80 (2002)). In an oxidative dehydrogenation process oxygen is added to the dehydrogenation reactor feed and reacts with the hydrogen produced during the dehydrogenation reaction. The hydrogen is converted to water, thereby removing it from the reaction zone and driving the thermodynamic equilibrium to higher alkane conversion values. The heat provided by the exothermic oxidation of hydrogen also can balance the heat required by the endothermic dehydrogenation reaction.

While the concept of oxidative dehydrogenation is not new, to date the process has not been commercialized for the large-scale production of light olefins. There are a number of drawbacks to the use of oxidative dehydrogenation as compared to standard catalytic dehydrogenation. First, addition of oxygen to the feed typically leads to a reduced selectivity to the desired olefin product. Formation of carbon oxides and oxygenated compounds through the undesirable partial combustion of the hydrocarbon feed can lead to lower feed utilization and more complex downstream separation requirements for oxidative dehydrogenation processes. Second, mixing of oxygen with the hydrocarbon feed presents a safety concern that is not present in conventional catalytic dehydrogenation processes. While these risks can be mitigated through the application of safe engineering and design principles and additional safety systems, these systems and procedures can increase the cost and complexity of the oxidative dehydrogenation process and in any case the risks cannot be entirely removed. Finally, presence of both exothermic oxidation and endothermic dehydrogenation reactions within the reactor presents a significant reactor design challenge with regard to the management of heat within the reactor.

It is believed that the most promising way at present to remove the thermodynamic limitation of olefins production is to employ membranes capable of removing hydrogen. Removal of hydrogen causes the chemical reaction to proceed to the right through the law of mass action, thereby achieving much higher conversions, up to 100 percent conversion.

Membranes have been explored that remove hydrogen and thereby allow higher yields of olefins to be achieved. For example, U.S. Pat. No. 3,290,406 describes the use of palladium alloy tubes to remove hydrogen formed during the dehydrogenation of ethane. Membranes made out of palladium or palladium alloys are the most widely explored membranes for hydrogen separations. There are numerous reports in the art of palladium or palladium alloy membranes demonstrating high hydrogen permeation rates and hydrogen selectivities.

However, issues remain to be solved before palladium or palladium alloy membranes can be used in an industrial setting, as pointed out in an article by Collins and coworkers entitled “Catalytic Dehydrogenation of Propane in Hydrogen Permselective Reactors” in Industrial Engineering and Chemistry Research, volume 35, pages 4398-4405 (1996). Collins and coworkers found that palladium membranes deactivated rapidly when placed in alkane dehydrogenation service. Their membranes failed because of a large deposition of coke on the surface of the palladium membrane.

U.S. Pat. No. 5,202,517, in the name of Ronald G. Minet, Theodore T. Tsotsis and Althea M. Champagnie, appears to describe a way to overcome the coking problems associated with palladium membranes by use of porous ceramic membranes impregnated on the surface with palladium or platinum which are contacted with a mixture of alkane and hydrogen. They state that the hydrogen in the feed is needed to suppress the formation of coke.

Another way to suppress the formation of coke on the surface of a hydrogen membrane reactor is to supply a source of oxygen to the membrane reactor in the form of pure dioxygen (diatomic oxygen), air, or steam. However, supplying diatomic oxygen or air to the feed side of a hydrogen membrane reactor would suffer from the same drawbacks associated with oxidative dehydrogenation, namely safety concerns and reduced selectivity to the desired olefin product through the formation of carbon oxides.

It is therefore a general object of the present invention to provide an improved process which overcomes the aforesaid problem of prior art methods for chemical conversion of volatile organic compounds to value added products using membrane reactors.

An improved method for conversion of alkanes to corresponding alkenes should provide better ways to introduce oxygen into the reactor in order to keep the membrane free of coke.

There is a need for membranes that are capable of simultaneously transporting hydrogen and oxygen. For the synthesis of alkenes from alkanes, it is important to carefully balance the rates of oxygen and hydrogen transport. High hydrogen transport is desirable to maximize the production of olefins. Oxygen transport needs to be sufficient to prevent coking problems but not so high as to oxidize the alkane feedstock and form carbon oxides.

Membrane compositions have been described for transport of electrons and hydrogen. Other membrane compositions have been described for conducting electrons and oxygen. Membranes composed of a single phase capable of simultaneous hydrogen and oxygen transport have been described. For example, membranes composed of a single mixed oxide for oxygen and hydrogen transport are described in an article entitled “Oxide Ion Conduction in Ytterbium-Doped Strontium Cerate” by N. Bonanos, B. Ellis and M. N. Mahmood in Solid State Ionics, vol. 28-30, pages 579-579 (1988). A single phase mixed membrane for alkane dehydrogenation is described in U.S. Pat. No. 5,821,185, U.S. Pat. No. 6,037,514 and U.S. Pat. No. 6,281,403 each in the name of in the name of James H. White, Michael Schwartz and Anthony F. Sammels and assigned to Eltron Research, Inc.

U.S. Pat. No. 6,332,964 in the name of Chieh Cheng Chen, Ravi Prasad, Terry J. Mazanec and Charles J. Besecker describes membranes composed of an electron conducting phase and an oxygen-conducting phase. There are distinct advantages associated with employing such a matrix as a membrane in a reactor. A known problem in a reactor of this type is the slow buildup of coke on the alkane side of the reactor. Using a membrane matrix that conducts oxygen ions may reduce, or even eliminate, coking problems. It is believed that oxygen can be transported from the air side of the membrane to the alkane side where it may reacts with coke precursors as they are formed on the membrane surface. Reaction of the coke precursors with oxygen also provides heat to fuel the endothermic dehydrogenation reaction. Another use for the oxygen that is transported through such a matrix is to react with hydrogen to produce heat, as is needed in steam reforming.

U.S. Pat. No. 6,066,307 in the name of Nitin Ramesh Keskar, Ravi Prasad and Christian Friedrich Gottzmann describes a process for preparing synthesis gas and hydrogen gas using a membrane reactor having two membranes, one membrane that is an oxygen conductor and the other membrane that is a proton conductor, to produce hydrogen gas and synthesis gas.

Dual phase membranes offer the potential to balance the rates of oxygen and hydrogen transport. If one phase is responsible for hydrogen transport and the other is responsible for oxygen transport, it would be possible to adjust the relative amounts of the two phases to maximize hydrogen transport while maintaining an oxygen transport rate sufficient to keep the membrane from coking but not so high as to oxidize the alkane feedstock. It is harder to achieve this balance in single-phase membranes. There is a need for dual phase membranes that conduct both hydrogen and oxygen in order to produce a membrane reactor that facilitates chemical conversions without deactivating too rapidly. For example, improvements in steam reforming and alkane dehydrogenation would be expected if these dual phase membranes were employed.

Alkene production technologies described above produce, along with the desired olefin products, a variety of unwanted or lower-value coproducts. For example, in the cracking of hydrocarbons to produce ethylene and propylene, coproducts such as methane, hydrogen, acetylene, and others are produced. Likewise, dehydrogenation of alkanes to produce the corresponding olefin also produces coproducts such as hydrogen, diolefins and acetylenics. Such coproducts make necessary one or more separation and purification steps so that a purified olefin product, suitable for downstream processing, can be obtained.

There is extremely wide scope for the design of such separation systems, limited only by the purity specifications of the final olefins product, technical feasibility, and economic viability. In a practical sense, however, such separation systems have nearly all contained at least the steps of compression of the olefin-containing reactor effluent stream, chilling and partial condensation of the compressed stream, and vapor/liquid separation wherein the liquid contains the olefin product and the vapor contains less valuable lighter gases.

For example and with respect to separation of products from the dehydrogenation of alkalis, U.S. Pat. No. 6,333,445 in the name of John V. O'Brien described a cryogenic separation system for the recovery of olefins from a dehydrogenation process. This process included compression of the dehydrogenation reactor effluent and multiple levels of chilling and subsequent vapor/liquid separation. U.S. Pat. No. 5,026,952 in the name of Heinz Bauer describes a process for recovering C₂+, C₃+ or C₄ hydrocarbons from a high-pressure stream containing these components and lighter components. This process included multiple chilling and vapor/liquid separation stages, as well as rectification and expansion steps. U.S. Pat. No. 5,177,293 in the name of Michael J. Mitariten and Norman H. Scott describes a process for the separation and recovery of product streams from a dehydrogenation reactor that uses a pressure swing adsorption process to concentrate the olefin product. This process also comprises the steps of compression of the dehydrogenation reactor effluent, chilling of the compressed effluent, and subsequent vapor/liquid separation.

With respect to the separation of products from a hydrocarbon cracking reactor, a variety of commercial processes are offered by various technology vendors, including ABB Lummus Global, Kellogg Brown & Root, Inc., Linde A. G., Stone and Webster, Inc., and Technip-Coflexip, among others. A summary of the commercially-available steam cracking and product purification technologies from these vendors has been published recently (Hydrocarbon Processing, March 2003, pp 96-98). While there are many and significant differences in the ethylene production and recovery processes offered by these vendors, it is clear to those skilled in the art that each of the ethylene production and purification processes contains at least the steps of compression of the furnace effluent, and the subsequent chilling and partial condensation of the compressed effluent to produce at least one olefin-rich liquid stream.

It is clear from the discussion above that the majority of separation processes which are necessary in the production of a purified olefin product from a reactor effluent comprise the steps of a) compression of the majority of the reactor effluent, b) chilling and partial condensation of the majority of the compressed reactor effluent stream, and subsequent separation of the resulting liquid and vapor phases to produce an olefin-rich liquid. These steps are common to essentially all light olefin-producing processes primarily because in the cases of dehydrogenation and steam cracking the reactor effluent exits the reactor at relatively low pressure, and because the subsequent purification of the desired olefin product is carried out largely through distillation and vapor/liquid separation. It would therefore be highly desirable to provide a reactor effluent stream which allows the subsequent steps of compression, chilling with partial condensation, and vapor/liquid separation to be carried out in a more energy- and capital-efficient manner.

Commercialized steam cracking processes utilize high temperature pyrolysis where these feeds are mixed with steam and heated to 700° C. to 900° C. During the process, less than 100 percent of the feed is converted per pass because of thermodynamic limitations and in order to maximize the yield of the desired olefin product. Complex and expensive separation equipment is used to recover the unreacted feed from the olefin products and byproducts. The unconverted feed is recycled where it is often remixed with fresh feed. This recycle introduces inefficiencies into the olefins process. It would be desirable to reduce this inefficiency by increasing per-pass conversion and thereby reducing or even eliminating recycles. Benefits of higher conversion olefins processes include reducing the size of equipment and capital employed in olefins manufacture, lowering the energy required to produce the olefin product, and elimination of large pieces of equipment devoted to separating the olefin product from unreacted alkane.

Other objects and advantages of the invention will become apparent upon reading the following detailed description and appended claims.

### SUMMARY OF THE INVENTION

In broad aspect, the present invention includes processes having as constituent parts high conversion membrane reactors, that provide a source of alkane monomer, and subsequent polymerization and/or oligomerization of the alkane, without passing products of the conversion through an alkane/alkene splitter. Equipment required for separation of alkene products from unreacted alkanes in conventional plants is eliminated because of the high alkane conversions provided in the membrane reactors. Particularly useful are flow reactors comprising dense membranes of multiphasic materials that provide independent, controllable, counter-current transport of hydrogen, electrons and oxygen.

In one aspect, the invention is a process for preparing a desired oligomer or polymer of a light alkene hydrocarbon having as constituent parts thereof a high conversion membrane reactor, that provides a source of monomer, and subsequent oligomerization and/or polymerization of the alkene, without passing products of the conversion through an alkane/alkene splitter, which process comprises a cooperating arrangement of the following steps: Providing a flow reactor comprising plurality of reaction zones each having at least one inlet for flow of fluid in contact with a transport membrane comprising at least one solid phase that demonstrates an ability to selectively convey hydrogen, and the same or another phase that demonstrates electronic conductivity, and at least one outlet for flow of effluent from the reaction zone; Introducing a feedstream comprising volatile organic compounds all or a portion of the reaction zones; Converting, in reaction zones at elevated temperatures, one or more volatile organic compound in the feedstream to products of conversion comprising a desired alkene containing from 2 to 6 carbon atoms, organic co-products and hydrogen; Permitting at least a portion of the hydrogen co-product to be selectively conveyed out of one or more of the reaction zones through the solid membrane, thereby obtaining a gaseous effluent from the reaction zone; Compressing at least a gaseous portion of the effluent from the reaction zone, cooling the compressed effluent gas to form a liquid fraction rich in products of conversion and a dihydrogen-rich gaseous fraction, and separating the fractions; Recovering from the liquid fraction a monomer stream containing the desired alkene as the predominant component which monomer is substantially free of organic compounds containing one or more carbon atoms than the desired alkene; and Reacting at least one alkene in the monomer stream to thereby provide a desired oligomer or polymer.

The volatile organic compounds in the feedstream may include one or more alkane hydrocarbon containing from 2 to 8 carbon atoms, and at least 75 percent of one alkane in the feedstream is converted to the desired alkene in the flow reactor. Where the effluent from the reaction zone comprises ethane, ethylene and acetylene, and the process beneficially comprises a treatment to convert acetylene to ethylene, thereby providing a treated monomer stream substantially free of acetylene. There after at least ethylene is reacted to form the desired polymer under conditions suitable for a gas phase, slurry, or solution polymerization process.

Where the alkane polymerization in the acetylene-free monomer stream is carried out in a gas phase process under conditions, at least 85 percent of the ethylene is reacted to polyethylene product on a once through basis. Where the polymerization is carried out in a slurry process operated under conditions, including pressures in a range upward from about 3,000 psi to about 5,000 psi, again at least 85 percent of the ethylene is reacted to polyethylene product on a once through basis. Where the polymerization is carried out in a solution process operated under conditions, including pressures in a range upward from about 3,000 psi to about 5,000 psi, yet again at least 85 percent of the ethylene is reacted to polyethylene product.

In another aspect, the invention provides a process wherein at least 85 percent of the ethylene in the monomer stream is reacted under conditions suitable for formation of alpha-olefins containing from about 6 to about 14 carbon atoms.

The process according to the invention may further comprise recovering from the polymerization a stream comprising ethane and/or unreacted ethylene, and introducing at least a portion of the recovered stream into one or more reaction zone in the high conversion membrane reactors.

Transport membranes of the invention include multiphasic solids formed by sintering homogeneous mixtures of powdered metals and metal oxide ceramics in particulate from. Useful powdered metals include at least one metal selected from the group consisting of silver, palladium, platinum, gold, rhodium, titanium, nickel, ruthenium, tungsten, and tantalum. Metal oxide ceramics used in formation of transport membranes of the invention advantageously comprise at least one mixed metal oxide having a perovskite structure or perovskite-like structure.

In another aspect, the invention is a process for preparing a desired oligomer or polymer of a light alkene hydrocarbon having as constituent parts thereof a high conversion membrane reactor, that provides a source of monomer, and a subsequent polymerization of the alkene, without passing products of the conversion through an alkane/alkene splitter, which process comprises a cooperating arrangement of elements. A flow reactor provides a plurality of reaction zones each having at least one inlet for flow of fluid in contact with a first side of a multiphasic, solid state, membrane comprising two or more phases bound to one another wherein at least one of the bound phases demonstrates an ability to selectively convey hydrogen, another phase demonstrates an ability to selectively convey oxygen ions between different gaseous mixtures, and one or more of the phases demonstrates electronic conductivity, and at least one outlet for flow of effluent from the reaction zone. A feedstream comprising volatile organic compounds, substantially free of dihydrogen and dioxygen, is introduced into all or a portion of the reaction zones. At elevated temperatures in the reaction zones, one or more volatile organic compound in the feedstream is converted to products of conversion comprising a desired alkene containing from 2 to 6 carbon atoms, organic co-products and hydrogen. At least a portion of the hydrogen co-product is selectively conveyed out of one or more of the reaction zones through the solid membrane to a second side thereof, thereby obtaining a gaseous effluent from the reaction zone that is characterized by a “Relative Hydrogen Index” value of less than 1.0. At least a gaseous portion of the effluent from the reaction zone is compressed. The compressed effluent gas is cooled thereby forming a liquid fraction rich in products of conversion and a dihydrogen-rich gaseous fraction. A monomer stream containing the desired alkene as the predominant component is recovered from the liquid fraction. The recovered monomer is substantially free of organic compounds containing one or more carbon atoms than the desired alkene. Alkenes in the monomer stream are polymerized to thereby provide the desired polymer product.

The Relative Hydrogen Index of the membrane reactor effluent is defined as a ratio of a deference between the total flow of hydrogen atoms and the flow of hydrogen atoms in the form of water in the effluent stream to the same difference for the feedstream. The Relative Hydrogen Index is represented by the equation:
RHI=(H_{T}−H_{W})_{E}/(H_{T}−H_{W})_{F}
where RHI is the Relative Hydrogen Index, the E subscript refers to the fow reactor effluent before any cooling or other processing, the F subscript refers to the reactor feed, H_{T} is the total flow of hydrogen atoms in the stream, and H_{W} is the flow of hydrogen atoms in the form of water vapor in the stream. For process for chemical conversion of volatile organic compounds to value added products according to the invention, membrane reactor effluents have an RHI of less than 1.0, and beneficially less than 0.75, by which savings of a significant amounts of energy are obtained in subsequent product recovery steps.

Such low-RHI reactor effluents can be produced by membrane reactors, which transport oxygen ions, hydrogen ions, or both oxygen and hydrogen ions. Examples are given which demonstrate that current technologies which convert alkanes to olefins, including steam cracking and dehydrogenation, produce effluents with an RHI equal to or greater than 1.0, while such membrane reactors can produce effluents with an RHI of less than 1.0.

In yet another aspect, the invention is a process for preparing a desired oligomer or polymer of a light alkene hydrocarbon having as constituent parts thereof a high conversion membrane reactor, that provides a source of monomer, and a subsequent polymerization of the alkene, without passing products of the conversion through an alkane/alkene splitter, which process comprises a cooperating arrangement of the following steps: Providing a flow reactor comprising plurality of reaction zones each having at least one inlet for flow of fluid in contact with a first side of a multiphasic, solid state, membrane comprising two or more phases bound to one another wherein at least one of the bound phases demonstrates an ability to selectively convey hydrogen, another phase demonstrates an ability to selectively convey oxygen ions between different gaseous mixtures, and one or more of the phases demonstrates electronic conductivity, and at least one outlet for flow of effluent from the reaction zone; Introducing a petroleum derived organic feedstream, substantially free of dihydrogen and dioxygen, selected from the group consisting of crude oil, distillate, vacuum gas oil, atmospheric gas oil, natural gas liquid, raffinate, naphtha and mixtures thereof, into all or a portion of the reaction zones; Converting one or more organic compound in the feedstream by breaking molecular bonds at elevated temperatures in the reaction zones, and thereby forming conversion products comprising alkene compounds containing from 2 to 6 carbon atoms, organic co-products and hydrogen; Permitting at least a portion of the hydrogen co-product to be selectively conveyed out of one or more of the reaction zones through the solid membrane to a second side thereof, thereby obtaining a gaseous effluent from the reaction zone that is characterized by a Relative Hydrogen Index value of less than 1.0; Compressing at least a gaseous portion of the effluent from the reaction zone, cooling the compressed effluent gas to form a liquid, rich in products of conversion, and a dihydrogen-rich gas; Partitioning the liquid, as by distillation, into an ethylene-rich fraction that is substantially free of organic compounds containing three and more carbon atoms, and another fraction that includes the organic compounds containing three and more carbon atoms; Recovering from the ethylene-rich fraction a monomer stream having an ethylene content in a range upward from about 70 percent by weight; and Polymerizing at least 80 percent of the ethylene in the monomer stream to thereby provide the desired polymer product. The conversions in the reaction zones are carried out by thermal, catalytic or hydrocracking methods.

In a further aspect of the invention, the fraction that includes organic compounds containing three and more carbon atoms is partitioned, as by distillation, to form a C₃ fraction comprising propylene, propadiene and methylacetylene which fraction is substantially free of organic compounds containing four or more carbon atoms, and a residue fraction that includes the organic compounds containing four or more carbon atoms. The C₃ fraction is treated to convert propadiene and/or methylacetylene to propylene and thereby form a resulting stream having a propylene content in a range upward from about 70 percent by weight. Beneficially, at least 80 percent of the propylene in the resulting stream is polymerized to thereby provide the desired polymer product.

Flow reactors of the invention advantageously comprise dense membranes which transport oxygen ions or hydrogen ions or oxygen and hydrogen ions at conditions suitable for the production of olefins. These membrane flow reactors typically are operated at temperatures in a range downward from about 1000° C. Flow conditions are controlled to provide effluents from the reaction zones of the membrane flow reactors at pressures in a range downward from about 450 psia.

### BRIEF DESCRIPTION OF THE INVENTION

The present invention allows elimination of equipment associated with alkane/alkene separation and thereby direct coupling of alkane production with polymerization processes.

These results unexpectedly showed that it is possible to break one of the principal business paradigms of the olefins industry, where one large mega-scale olefins plant supplies several polyolefins plants. This allows smaller scale olefins plants to be built where they are needed to supply a polyolefin plant.

Flow reactors of the invention provide high feedstock conversion levels together with yields of olefins from the membrane reactors that are significantly higher than that of conventional steam cracking processes. The benefits of simultaneous high olefin yield and high feedstock conversion possible with membrane reactors results in capital and operating costs for olefins production that are significantly lower than high temperature steam cracking.

Transport membranes useful in flow reactors of the invention comprise a sintered homogenous mixture of a ceramic composition and a metal that demonstrates an ability to selectively convey hydrogen. Useful metals include palladium, niobium, tantalum, vanadium, or zirconium or a binary mixture of palladium with another metal such as niobium, silver, tantalum, vanadium, or zirconium. Membrane materials advantageously comprise at least one mixed metal oxide having a perovskite structure or perovskite-like structure. See U.S. Pat. No. 6,569,226 in the name of Stephen E. Dorris, Tae H. Lee, and Uthamalingam Balachandran, the entire disclosure of which is incorporated herein by reference. Membranes for use in flow reactors of the invention may be in sheet form or tubular form or honeycomb form, such as illustrated in U.S. Pat. No. 5,356,728 in the name of Uthamalingam Balachandran, Roger B. Poeppel, Mark S. Kleefisch, Thaddeus P. Kobylinski and Carl A. Udovich, the entire disclosure of which is incorporated herein by reference.

Materials known as “perovskites” are a class of materials which have an X-ray identifiable crystalline structure based upon the structure of the mineral perovskite, CaTiO₃. In its idealized form, the perovskite structure has a cubic lattice in which a unit cell contains metal ions (A) at the corners of the cell, another metal ion (B) in its center and oxygen ions at the center of each cube face. In known crystalline materials of the perovskite class, the unit cell repeats, without interruption throughout a crystal, in all three orthogonal directions of a suitably oriented rectangular coordinate system. This cubic lattice is identified as an ABO₃-typestructure where A and B represent metal ions. In the idealized form of perovskite structures, generally, it is required that the sum of the valences of A ions and B ions equal 6, as in the model perovskite mineral, CaTiO₃.

Many materials having the perovskite-type structure (ABO₃-type, i.e., where β is zero) have been described in recent publications including a wide variety of multiple cation substitutions on both the A and B sites said to be stable in the perovskite structure. Likewise, a variety of more complex perovskite compounds containing a mixture of A metal ions and B metal ions (in addition to oxygen) are reported. Materials and methods useful in dense ceramic membrane preparation of the invention are described in U.S. Pat. App. Pub. No.: US 2005/0222479 A1, which publication is hereby incorporated herein by reference for its disclosure relating to preparation of dense ceramic membranes. Other publications relating to dense ceramic membranes include, for example P. D. Battle et al., J. Solid State Chem., 76, 334 (1988); Y. Takeda et al., Z. Anorg., Allg. Chem., 550/541, 259 (1986); Y. Teraoka et al., Chem. Lett., 19, 1743 (1985); P. D. Battle et al., J. Solid State Chem., 76, 334 (1988); M. Harder and H. H. Muller-Buschbaum, Z. Anorg. Allg. Chem., 464, 169 (1980); C. Greaves et al., Acta Cryst., B31, 641 (1975).

Any of a variety of methods may be used to make inorganic crystalline materials as described herein above. According to the present invention, useful multiphasic systems advantageously comprise two or more phases bound to one another. At least one of the bound phases demonstrates an ability to selectively convey hydrogen; another phase demonstrates an ability to selectively convey oxygen ions between different gaseous mixtures; and one or more of the phases demonstrates electronic conductivity.

Processes of the present invention include preparing ethylbenzene or substituted derivatives thereof from ethane and benzene, or ethane and substituted benzenes. Ethylbenzene and/or substituted ethylbenzenes are useful for preparing styrene and substituted styrenes, which are intermediate materials for polystyrene plastics.

Processes of this invention may also comprise as constituent parts thereof dehydrogenating ethane to produce a monomer stream containing ethylene as a predominant component, and thereafter, alkylating benzene or substituted benzene with the ethylene containing monomer stream to yield ethylbenzene or substituted ethylbenzene.

Dehydrogenation elements in this invention may comprises contacting, for example, in a high conversion membrane reactor an ethane containing feedstream with a catalytic amount of a dehydrogenation catalyst in a dehydrogenation zone. Contacting is conducted under reaction conditions such that a dehydrogenation product stream containing predominantly ethylene and unreacted ethane is formed. Typically, dehydrogenation catalysts comprise a mordenite zeolite and, optionally, a metal component selected from the group consisting of gallium, zinc, ruthenium, osmium, rhodium, iridium, palladium and platinum.

Typically, a monomer stream containing the desired concentration of ethylene is recovered from a condensed liquid fraction of the dehydrogenation product stream. In one aspect of the invention, the dehydrogenation product stream, with essentially no further purification or separation, and a benzene co-feed are contacted with a catalytic amount of an alkylation catalyst in an alkylation zone under reaction conditions such that ethylbenzene is produced. Alternatively, a substituted benzene may be employed in the alkylation zone to produce a substituted ethylbenzene product. See, for example, U.S. Pat. No. 5,430,211 in the name of Randall F. Pogue, Juan M. Garces, Timothy M. May, and Andrew Q Campbell.

As indicated above, alkenes from the monomer stream may be polymerized or oligomerized to thereby provide the desired product. Any method suitable for a monomer stream having an alkene content in a range upward from about 70 percent by weight. For example, a monomer stream comprising ethylene as a predominate alkene may be polymerized or oligomerized into polyethylene or alpha olefins either by solution, slurry or gas phase methods.

Generally the pressure in the polymerization reactor for a solution process may be in a range upward from about 3,000 psi to about 10,000 psi, typically in a range from 3,000 psi to 5,000 psi. This moderate pressure is necessary to maintain the monomers and co-monomers in solution even at the solution's high reaction temperature.

In solution and/or slurry polymerization processes, ethylene is dissolved in a liquid hydrocarbyl medium (e.g., alkyl and/or an aromatic hydrocarbon containing from about 5 to about 10 carbon atoms). The solution is contacted, as by mixing, with a catalyst. Useful catalysts include conventional Ziegler-Natta type catalyst, a metallocene type catalyst as used by Exxon, a constrained geometry catalyst as used by Dow or it could contain novel ligands such as phosphinimine ligands. Depending on the temperature, the process may be a slurry process (polymer precipitates from solution) as are disclosed for example in patents to Phillips (typically at temperatures below about 100° C.) or the polymer may remain in solution (at temperature from about 180° C. to about 300° C.) as described in patents in the name of DuPont Canada, Novacor and Nova Chemicals Ltd. Useful catalysts for oligomerization may also be nickel, zirconium, chromium or titanium based.

In solution and slurry polymerization processes, the catalyst is deactivated and remains in the polymer. Polymer products are then separated from the liquid solution, which is generally heated to remove residual unreacted alkane and alkene hydrocarbons (e.g., ethylene and ethane). These are typically recycled, to the high conversion membrane reactors.

Formation of alpha-olefins containing from about 6 to about 14 carbon atoms, in a dual displacement loop ethylene/tri-lower alkyl chain growth process, is disclosed in U.S. Pat. No. 4,935,569 in the name of Alvin E. Harkins, and Layne W. Summers, the entire disclosure of which is incorporated herein by reference.

Alkene polymerization may also employ a gas phase polymerization, typically conducted at temperatures in a range upward from about 80° C. to about 115° C. and at pressures from about atmospheric to about 150 psig.

In another aspect of the present invention, alkenes from the monomer stream are oligomerized to thereby provide the desired product. For example, ethylene can be condensed to form higher alpha olefins such as butene, hexene and octene although higher alpha olefins, containing up to about 20 carbon atoms, may be produced. Ethylene is contacted with an oligomerization catalyst typically comprising of an aluminum alkyl (e.g., trimethyl aluminum or tri ethyl aluminum) or an aluminum complex represented by the formula
(R)₂AlO(RAlO)ₘAl(R)₂
wherein each R is independently selected from the group consisting of hydrocarbyl radicals containing from 1 to about 20 carbon atoms, and m is a number from 0 to 50. Advantageously, R is an alkyl radical containing from 1 to 4 carbon atoms, and m is a number from 5 to 30. Growing ethylene alpha olefins are typically recycled through a reaction or chain growth zone to grow the alpha olefins, as a result there is generally a statistical distribution of alpha olefins in the resulting products. Therefore, the alpha olefins are separated, typically by distillation, to purify the different alpha olefins. Generally, processes for oligomerization of ethylene are conducted under pressures from about 2,000 psig to 5,000 psig, preferably 2,000 psig to 3,500 psig, and at temperatures from about 90° C. to about 180° C. (See, for example, U.S. Pat. No. 4,935,569 in the name of Alvin E. Harkins and Layne W. Summers.)

In other ethylene oligomerizations, the catalyst comprises transition metal complexes and alkylaluminum compounds represented by the formula
AlR_{y}X_{3-y}
as co-catalysts where R is an alkyl radical containing from 1 to 8 carbon atoms, preferably from 1 to 4 carbon atoms, X is a halogen, preferably chlorine or bromine, and y is an integer from 1 to 3. Titanium, zirconium, hafnium, chromium, nickel or molybdenum could be used as active components of useful complex catalysts. These processes require milder reaction conditions, including pressures from 15 psig to 1500 psig and temperatures from 0° C. to 150° C.

Other alkene polymerization catalysts and process for the polymerization have been described, for example in U.S. Pat. No. 5,321,107 in the name of Toshiyuki Tsutsui, Kazunori Okawa, and Akinori Toyota.

The following examples will serve to illustrate certain specific embodiments of the herein-disclosed invention. These examples should not, however, be construed as limiting the scope of the novel invention, as there are many variations which may be made thereon without departing from the spirit of the disclosed invention, as those of skill in the art will recognize.

### EXAMPLES OF THE INVENTION

General

The present invention is described by way of a number of relevant examples. These examples show that suitable membrane-based reactors, operating at low temperatures and high conversions, require significantly lower capital costs and operating expenses than conventional high temperature steam cracking.

### Comparative Example A

This example describes a conventional steam cracking process for production of polymer grade ethylene. High temperatures and low conversions typical of conventional steam cracking are employed in this comparative example. Yield data was obtained from steam cracker operations at 71 percent per-pass ethane conversion, dilution steam ratio of 0.33 wt/wt steam:hydrocarbon, and coil outlet temperature of 1010° C.

Effluent from the conventional steam cracking furnaces is subjected to a series of processing steps, including compression, deethanization, acetylene conversion, demethanization, and alkene/alkane splitting. Flowrates in this comparative example were sized to produce 300,000 metric tons of ethylene per year. Feeds into the furnaces include 46 te/hr ethane, 19 te/hr of ethane recycled from the C₂ splitter and 14 te/hr of steam, to provide 65 te/hr of furnace effluent.

Those skilled in the art will recognize that such a steam cracker is several times smaller than a typical world-scale steam cracker. This was done to show the flowrates associated with producing approximately enough ethylene to supply a single, world-scale polyethylene plant.

### Example 1

This example demonstrates the temperatures and conversions associated with membrane-based olefins reactors. Temperatures less than 1010° C. and conversion above 75 percent were obtained from the membrane-reactor in this example.

A multiphasic, solid state, hydrogen, electrons and oxygen transport membrane was fabricated from cerium gadolinium oxide and silver/palladium (CGO/(Ag/Pd)) as follows:

a) A batch of cerium gadolinium oxide powder, obtained from Rhodia, was heated in air to 1000° C. and held at that temperature for one hour. The powder was then sifted with a 60 mesh filter.

b) 1.93 g of the sifted cerium gadolinium oxide powder was mixed with 2.13 g of palladium/silver (70/30) flake, obtained from Degussa Corporation, for 30 minutes in a mortar and pestle.

c) Approximately 6 g of the mixture was loaded into a cylindrical dye (1.25 inch diameter) and compressed to 26,000 lbs. using a Carver Laboratory Press (Model #3365).

d) The CGO/(Ag/Pd) disc was sintered by heating in air to 1300° C. and holding at that temperature for 4 hours.

The sintered membrane was placed between two gold rings and heated to 900° C. at 0.5° C./minute. The sintered membrane was sealed with gold rings into a two-zone reactor. While a disc reactor was used in this example, the principles of operation are the same for a tube or cylindrical shaped reactor.

The reactor was heated to 800° C. under nitrogen. One side of the reactor was exposed to air and the other side exposed to ethane and steam (ethane and steam in a 1:1 weight ratio). The product from the hydrocarbon side was analyzed by gas chromatography. The carbon weight percent composition of the product is presented in Table I.

The selectivity for ethylene in this example was at least 88 percent at reactor temperatures of about 885° C. The membrane was studied for 600 hours in ethane/steam service and was stable.

TABLE I

| Conversion | 88.2% |
|---|---|
| Temperature | 885° C. |
| Material | CGO/(Ag/Pd) |
| Sweep | Air |
| Component | Selectivity |
| CO | 0.5 |
| Methane | 8.8 |
| Ethane | |
| Ethylene | 81.8 |
| Acetylene | 2.57 |
| Propane | |
| Propylene | 1.12 |
| Propadiene | 3.19 |
| Pentenes | 2.02 |

### Example 2

This example demonstrates the large benefits of a lower flowrates in the downstream processing of the effluent from a membrane-based reactor. It will further show that significant capital savings are expected from the elimination of equipment for ethylene/ethane splitting.

Using the results shown in Table I, calculations were performed to simulate a plant capable of producing 300,000 metric tons of ethylene per year using membrane reactors.

The results of these scaling calculations required feed into the membrane reactor of 43 te/hr of ethane and 7 te/hr of ethane recycled from the polyolefins plant, to provide 50 te/hr of reactor effluent.

Table I: Selectivity Pattern of Ethane from Membrane Reactor

Several benefits arise from the ability of the membrane reactors to operate at higher conversions. Approximately 7 less feedstock was required for the higher conversion process. Feedstock costs are one of the most significant components of the variable cost of ethylene production and this reduction represents a significant improvement in the economics of ethylene production.

The recycle rate of ethane was significantly reduced in the higher conversion process of the present invention. This indicates that the overall efficiency of the high conversion process is higher since the recycle represents a significantly lower portion of material going to the reactor in the high conversion process. Note that the ethane per-pass conversion used in this example was 88.2 percent and was chosen to match the experimental results shown in Table I.

Significantly higher conversions than those shown in Table I have been observed in the laboratory and consequently it is possible to nearly eliminate the recycle with the present invention.

Another significant component in the variable cost of olefins production is energy cost. The flow reactor gas feed rate and consequently the energy required to compress the gas was approximately 23 percent lower for the higher conversion process of this invention. This represents an enormous improvement in the economics of ethylene production.

The reduced flowrate of reactor effluent being processed in the back-end of the high conversion process of the invention reduces the size and capital cost of the equipment in this section of the plant. Using an equipment capital scaling factor of 0.65 (see for example Peters, M. S., and Timmerhaus, K. D. in “Plant Design and Economics for Chemical Engineers”, McGraw Hill (1991) or Garrett, D. E. in “Chemical Engineering Economics”, Van Nostrand Reinhold (1989)), a reduction in capital cost for the back-end equipment for the high conversion process of approximately 16 percent is expected. Combined with the reduced, back-end refrigeration energy requirements associated with lower flowrates for the high conversion process, the results indicate an unexpectedly large benefit for the high conversion process.

Additional capital savings are expected since the ethylene/ethane mixture obtained from the compression, separation, and acetylene conversion section of the high conversion plant does not need to pass through a C₂ splitter before the ethylene product is transferred for further processing. This is because the olefin to paraffin ratio in the present invention is significantly higher than that for conventional steam cracking. The capital costs savings associated with removing the C₂ splitter from the process are expected to be significant, on the order of $20 million, since a significant part of the refrigeration system in a conventional plant is associated with operating the C₂ splitter.

The high conversion process of the invention for this example was sized to produce enough ethylene to completely supply a 300,000 metric ton per year polyethylene plant. Those skilled in the art will recognize that processes of the invention represents a significant breakthrough in the business paradigm of the olefins industry, where one large mega-scale olefins plant supplies several polyolefins plants.

That which is claimed is:

1. A process for preparing a desired oligomer or polymer of a light alkene hydrocarbon having as constituent parts thereof a high conversion membrane reactor, that provides a source of monomer, and subsequent oligomerization and/or polymerization of the alkene, without passing products of the conversion through an alkane/alkene splitter, which process comprises a cooperating arrangement of the following steps:
(A) Providing a flow reactor comprising plurality of reaction zones each having at least one inlet for flow of fluid in contact with a transport membrane comprising at least one solid phase that demonstrates an ability to selectively convey hydrogen, and the same or another phase that demonstrates electronic conductivity, and at least one outlet for flow of effluent from the reaction zone;
(B) Introducing a feedstream comprising volatile organic compounds into all or a portion of the reaction zones;
(C) Converting, in reaction zones at elevated temperatures, one or more volatile organic compound in the feedstream to products of conversion comprising a desired alkene containing from 2 to 6 carbon atoms, organic co-products and hydrogen;
(D) Permitting at least a portion of the hydrogen co-product to be selectively conveyed out of one or more of the reaction zones through the solid membrane, thereby obtaining a gaseous effluent from the reaction zone;
(E) Compressing at least a gaseous portion of the effluent from the reaction zone, cooling the compressed effluent gas to form a liquid fraction rich in products of conversion and a dihydrogen-rich gaseous fraction, and separating the fractions;
(F) Recovering from the liquid fraction a monomer stream containing the desired alkene as the predominant component which monomer is substantially free of organic compounds containing one or more carbon atoms than the desired alkene; and
(G) Reacting at least one alkene in the monomer stream to thereby provide a desired oligomer or polymer.

2. The process according to claim 1 wherein the volatile organic compounds in the feedstream include one or more alkane hydrocarbon containing from 2 to 8 carbon atoms, and at least 75 percent of one alkane in the feedstream is converted to the desired alkene in the flow reactor.

3. The process according to claim 1 wherein the effluent from the reaction zone comprises ethane, ethylene and acetylene, and the process further comprises a treatment to convert acetylene to ethylene, thereby providing a treated monomer stream substantially free of acetylene.

4. The process according to claim 3 wherein at least ethylene is reacted to form the desired polymer under conditions suitable for a gas phase, slurry, or solution polymerization process.

5. The process according to claim 3 wherein the polymerization is carried out in a gas phase process under conditions whereby at least 85 percent of the ethylene is reacted to polyethylene product on a once through basis.

6. The process according to claim 3 wherein the polymerization is carried out in a slurry process operated under conditions, including pressures in a range upward from about 3,000 psi to about 5,000 psi, whereby at least 85 percent of the ethylene is reacted to polyethylene product on a once through basis.

7. The process according to claim 3 wherein the polymerization is carried out in a solution process operated under conditions, including pressures in a range upward from about 3,000 psi to about 5,000 psi, whereby at least 85 percent of the ethylene is reacted to polyethylene product.

8. The process according to claim 3 wherein at least 85 percent of the ethylene in the monomer stream is reacted under conditions suitable for formation of alpha-olefins containing from about 6 to about 14 carbon atoms.

9. The process according to claim 3 which further comprises recovering from the polymerization a stream comprising ethane and/or unreacted ethylene, and introducing at least a portion of the recovered stream into one or more reaction zone in the high conversion membrane reactors.

10. The process according to claim 1 wherein the transport membrane is a multiphasic solid formed by sintering homogeneous mixtures of powdered metals and metal oxide ceramics in particulate from.

11. The process according to claim 10 wherein the powdered metal comprises at least one metal selected from the group consisting of silver, palladium, platinum, gold, rhodium, titanium, nickel, ruthenium, tungsten, and tantalum.

12. The process according to claim 10 wherein the ceramic comprise at least one mixed metal oxide having a perovskite structure or perovskite-like structure.

13. A process for preparing a desired oligomer or polymer of a light alkene hydrocarbon having as constituent parts thereof a high conversion membrane reactor, that provides a source of monomer, and a subsequent polymerization of the alkene, without passing products of the conversion through an alkane/alkene splitter, which process comprises a cooperating arrangement of the following steps:
(A) Providing a flow reactor comprising plurality of reaction zones each having at least one inlet for flow of fluid in contact with a first side of a multiphasic, solid state, membrane comprising two or more phases bound to one another wherein at least one of the bound phases demonstrates an ability to selectively convey hydrogen, another phase demonstrates an ability to selectively convey oxygen ions between different gaseous mixtures, and one or more of the phases demonstrates electronic conductivity, and at least one outlet for flow of effluent from the reaction zone;
(B) Introducing a feedstream comprising volatile organic compounds, substantially free of dihydrogen and dioxygen, into all or a portion of the reaction zones;
(C) Converting, in reaction zones at elevated temperatures, one or more volatile organic compound in the feedstream to products of conversion comprising a desired alkene containing from 2 to 6 carbon atoms, organic co-products and hydrogen;
(D) Permitting at least a portion of the hydrogen co-product to be selectively conveyed out of one or more of the reaction zones through the solid membrane to a second side thereof, thereby obtaining a gaseous effluent from the reaction zone that is characterized by a Relative Hydrogen Index value of less than 1.0; and
(E) Compressing at least a gaseous portion of the effluent from the reaction zone, cooling the compressed effluent gas to form a liquid fraction rich in products of conversion and a dihydrogen-rich gaseous fraction, and separating the fractions;
(F) Recovering from the liquid fraction a monomer stream containing the desired alkene as the predominant component which monomer is substantially free of organic compounds containing one or more carbon atoms than the desired alkene; and
(G) Polymerizing alkenes from the monomer stream to thereby provide the desired polymer product.

14. The process according to claim 13 wherein the volatile organic compounds in the feedstream include one or more alkane hydrocarbon containing from 2 to 8 carbon atoms, and at least 75 percent of one alkane in the feedstream is converted to the desired alkene in the flow reactor.

15. The process according to claim 13 wherein the transport membrane is a multiphasic solid formed by sintering homogeneous mixtures of powdered metals and metal oxide ceramics in particulate from.

16. The process according to claim 15 wherein the powdered metal comprises at least one metal selected from the group consisting of silver, palladium, platinum, gold, rhodium, titanium, nickel, ruthenium, tungsten, and tantalum.

17. The process according to claim 15 wherein the ceramic comprise at least one mixed metal oxide having a perovskite structure or perovskite-like structure.

18. The process according to claim 15 wherein the effluent from the reaction zone comprises ethane, ethylene and acetylene, and the process further comprises a treatment to convert acetylene to ethylene, thereby providing a treated monomer stream substantially free of acetylene.

19. The process according to claim 18 which further comprises recovering from the polymerization a stream comprising ethane and/or unreacted ethylene, and introducing at least a portion of the recovered stream into one or more reaction zone in the high conversion membrane reactors.

20. A process for preparing a desired oligomer or polymer of a light alkene hydrocarbon having as constituent parts thereof a high conversion membrane reactor, that provides a source of monomer, and a subsequent polymerization of the alkene, without passing products of the conversion through an alkane/alkene splitter, which process comprises a cooperating arrangement of the following steps:
(A) Providing a flow reactor comprising plurality of reaction zones each having at least one inlet for flow of fluid in contact with a first side of a multiphasic, solid state, membrane comprising two or more phases bound to one another wherein at least one of the bound phases demonstrates an ability to selectively convey hydrogen, another phase demonstrates an ability to selectively convey oxygen ions between different gaseous mixtures, and one or more of the phases demonstrates electronic conductivity, and at least one outlet for flow of effluent from the reaction zone;
(B) Introducing a petroleum derived organic feedstream, substantially free of dihydrogen and dioxygen, selected from the group consisting of crude oil, distillate, vacuum gas oil, atmospheric gas oil, natural gas liquid, raffinate, naphtha and mixtures thereof, into all or a portion of the reaction zones;
(C) Converting one or more organic compound in the feedstream by breaking molecular bonds at elevated temperatures in the reaction zones, and thereby form conversion products comprising alkene compounds containing from 2 to 6 carbon atoms, organic co-products and hydrogen;
(D) Permitting at least a portion of the hydrogen co-product to be selectively conveyed out of one or more of the reaction zones through the solid membrane to a second side thereof, thereby obtaining a gaseous effluent from the reaction zone that is characterized by a Relative Hydrogen Index value of less than 1.0; and
(E) Compressing at least a gaseous portion of the effluent from the reaction zone, cooling the compressed effluent gas to form a liquid, rich in products of conversion, and a dihydrogen-rich gas;
(F) Partitioning the liquid, as by distillation, into an ethylene-rich fraction that is substantially free of organic compounds containing three and more carbon atoms, and another fraction that includes the organic compounds containing three and more carbon atoms;
(G) Recovering from the ethylene-rich fraction a monomer stream having an ethylene content in a range upward from about 70 percent by weight; and
(H) Polymerizing at least 80 percent of the ethylene in the monomer stream to thereby provide the desired polymer product.

21. The process according to claim 20 wherein the conversion in the reaction zones is carried out by thermal, catalytic or hydrocracking methods.

22. The process according to claim 20 which further comprises:
(i) Partitioning the fraction that includes organic compounds containing three and more carbon atoms, as by distillation, to form a C₃ fraction comprising propylene, propadiene and methylacetylene which fraction is substantially free of organic compounds containing four or more carbon atoms, and a residue fraction that includes the organic compounds containing four or more carbon atoms;
(ii) Treating the C₃ fraction to convert propadiene and/or methylacetylene to propylene and thereby form a resulting stream having a propylene content in a range upward from about 70 percent by weight; and
(iii) Polymerizing at least 80 percent of the propylene in the resulting stream to thereby provide the desired polymer product.

23. The process according to claim 22 which further comprises recovering from the polymerization stream comprising propane and/or unreacted propylene, and introducing at least a portion of the recovered stream into one or more reaction zone in the high conversion membrane reactors.

24. The process according to claim 20 wherein said reactor comprises a dense membrane which transports oxygen ions, hydrogen ions, or oxygen and hydrogen ions at conditions suitable for the production of olefins.

25. The process according to claim 20 wherein the membrane flow reactors are operated at temperatures in a range downward from about 1000° C.

26. The process according to claim 25 wherein effluents from the reaction zones of the membrane flow reactors are maintained at pressures in a range downward from about 450 psia.

27. A process for preparing a desired oligomer or polymer of a light alkene hydrocarbon having as constituent parts thereof a high conversion membrane reactor, that provides a source of monomer, and a subsequent polymerization of the alkene, without passing products of the conversion through an alkane/alkene splitter, which process comprises a cooperating arrangement of the following steps:
(A) Providing a flow reactor comprising plurality of reaction zones each having at least one inlet for flow of fluid in contact with a first side of a multiphasic, solid state, membrane comprising two or more phases bound to one another wherein at least one of the bound phases demonstrates an ability to selectively convey hydrogen, another phase demonstrates an ability to selectively convey oxygen ions between different gaseous mixtures, and one or more of the phases demonstrates electronic conductivity, and at least one outlet for flow of effluent from the reaction zone;
(B) Introducing a petroleum derived organic feedstream, substantially free of dihydrogen and dioxygen, selected from the group consisting of crude oil, distillate, vacuum gas oil, atmospheric gas oil, natural gas liquid, raffinate, naphtha and mixtures thereof, into all or a portion of the reaction zones;
(C) Converting one or more organic compound in the feedstream by breaking molecular bonds at elevated temperatures in the reaction zones, and thereby form conversion products comprising alkene compounds containing from 2 to 6 carbon atoms, organic co-products and hydrogen;
(D) Permitting at least a portion of the hydrogen co-product to be selectively conveyed out of one or more of the reaction zones through the solid membrane to a second side thereof, thereby obtaining a gaseous effluent from the reaction zone that is characterized by a Relative Hydrogen Index value of less than 1.0; and
(E) Compressing at least a gaseous portion of the effluent from the reaction zone, cooling the compressed effluent gas to form a liquid, rich in products of conversion, and a dihydrogen-rich gas;
(F) Partitioning the liquid, as by distillation, into an ethylene-rich fraction that is substantially free of organic compounds containing three and more carbon atoms, and another fraction that includes the organic compounds containing three and more carbon atoms;
(G) Partitioning the fraction that includes organic compounds containing three and more carbon atoms, as by distillation, to form a C₃ fraction comprising propylene, propadiene and methylacetylene which fraction is substantially free of organic compounds containing four or more carbon atoms, and a residue fraction that includes the organic compounds containing four or more carbon atoms;
(H) Treating the C₃ fraction to convert propadiene and/or methylacetylene to propylene and thereby form a resulting stream having a propylene content in a range upward from about 70 percent by weight; and
(I) Polymerizing at least 80 percent of the propylene in the resulting stream to thereby provide the desired polymer product.

28. The process according to claim 27 wherein the conversion in the reaction zones is carried out by thermal, catalytic or hydrocracking methods.
